(19) United States
(12) Patent Application Publication
(10) Pub. No.: US 2005/0005309 A1
(43) Pub. Date: Jan. 6, 2005

(54) Method of using animal models to predict adverse drug reactions

(76) Inventors: Glenn H. Cantor, Hopewell Township, NJ (US); Martin Geppert, Ilford (GB)

Correspondence Address: PHARMACIA & UPJOHN, 301 HENRIETTA ST, 0228-32-LAW, KALAMAZOO, MI, 49007, US

(21) Appl. No.: 10/689,225

(22) Filed: Oct. 20, 2003

Related U.S. Application Data
(60) Provisional application No. 60/421,950, filed on Oct. 29, 2002.

(51) Int. Cl. A01K 67/027

(52) U.S. Cl. 800/3; 800/14

(57) ABSTRACT

The invention relates generally to the process of target validation in the pharmaceutical industry. A process for validating molecular targets is disclosed.

### CROSS REFERENCE TO RELATED APPLICATION

This application claims the benefit of the following provisional application; Ser. No. 60/421,950, filed Oct. 29, 2002, under 35 U.S.C. 119(e)(1).

### FIELD OF THE INVENTION

The invention relates generally to the process of target validation in the pharmaceutical industry.

### BACKGROUND

The pharmaceutical industry abandons many compounds in the early stages of clinical development due to the discovery of toxic effects in humans. Because of the years of animal testing that precedes testing in humans, this is an expensive loss. To reduce expenses, it is desirable to identify compounds with adverse effects early and eliminate them from further development. Additionally, if adverse effects could be identified earlier, it may be possible to design safer compounds at an earlier stage, thus significantly accelerating the time required to develop drugs that are both safe and efficacious. One early stage for identifying potential problems is during the selection of molecular targets that will be inhibited or stimulated by new drugs. This process is termed “target validation.”

One problem with the present path of drug development is that the test animals are kept in uniform, carefully controlled environments. While this allows reproducible and scientifically accurate data collection, it does not accurately mimic the human situation, in which people are exposed to concurrent illnesses, psychological stresses, traumatic injury, nutritional problems, and other common physiologic perturbations.

Here, we propose a process in which target validation can be efficiently tested in animals using defined perturbations and a clearly defined, measurable, and rapid method to analyze the results.

### Literature Cited

1. Brayton, C., M. Justice, and C. A. Montgomery. 2001. Evaluating mutant mice: Anatomic pathology. Veterinary Pathology 38:1-19.
2. Campbell, K. H. S., J. McWhir, W. A. Ritchie, & I. Wilmut, 1996. Sheep Cloned by Nuclear Transfer from a Cultured Cell Line, Nature 380: 64-66.
3. Gavaghan, C. L., E. Holmes, E. Lenz, I. D. Wilson, and J. K. Nicholson. 2000. An NMR-based metabonomic approach to investigate the biochemical consequences of genetic strain differences: application to the C57BL10J and Alpk: ApfCD mouse. FEBS Letters 484: 169-174.
4. Griebel, G., J. Simiand, R. Steinberg, M. Jung, D. Gully, P. Roger, M. Geslin, B. Scatton, J. P. Maffrand, and P. Soubrie. 2002. 4-(2-Chloro-4-methoxy-5-methylphenyl)-N-[(1S)-2-cyclopropyl-1-(3-fluoro-4-methylphenyl)ethyl]5-methyl-N-(2-propynyl)-1,3-thiazol-2-amine hydrochloride (SSR125543A), a potent and selective corticotrophin-releasing factor(1) receptor antagonist. II. Characterization in rodent models of stress-related disorders. Journal of Pharmacology and Experimental Therapeutics 301:333-345.
5. Houdebine, L. M.; Transgenic Animal Generation and Use, Harwood Academic Press, 1997.
6. Robertson, D. G., M. D. Reily, J. C. Lindon, E. Holmes, and J. K. Nicholson. 2002. Metabonomic technology as a tool for rapid throughput in vivo toxicity screening, p. 583-626. In J. P. Vanden Heuvel, G. H. Perdew, W. B. Mattes, and W. F. Greenlee (ed.), Comprehensive Toxicology, vol. 14. Elsevier, Amsterdam.
7. Robertson, D. G., E. M. Urda, M. A. Breider, and R. M. Gauthier. 1998. Evaluation of hepatic toxicity of seven-day repeated-dose glutathione-depleting regimens in rats. Toxicology Methods 8:233-244.
8. Warren, T. K., K. A. Mitchell, and B. P. Lawrence. 2000. Exposure to 2,3,7,8-tetrachlorodibenzo-p-dioxin (TCDD) suppresses the humoral and cell-mediated immune responses to influenza A virus without affecting cytolytic activity in the lung. Toxicological Sciences 56: 114-123.
9. Wilmut, I., A. E. Schnieke, J. McWhir, A. J. Kind, & K. H. S. Campbell, 1997. Viable Offspring Derived From Fetal and Adult Mammalian Cells. Nature, 385: 810-813.

### Books

10. Sundberg, J. P., and D. Boggess. 2000. Systematic approach to evaluation of mouse mutations, 1 ed. CRC Press, Boca Raton, Fla.
11. Tymms, M. J., and I. Kola. 2001. Gene knockout protocols. Humana Press, Totawa, N.J.
12. Ward, J. M., J. F. Mahler, R. R. Maronpot, and J. P. Sundberg. 2000. Pathology of genetically engineered mice, 1 ed. Iowa State University Press, Ames, Iowa.

### Patents

13. U.S. Pat. No. 5,523,222

### SUMMARY OF THE INVENTION

The invention comprises in one aspect a process for predicting adverse responses to drugs against a target gene by assessing the responses of animal models, comprising:
(a) providing a genetically engineered non-human mammal. wherein said mammal exhibits either over-expression or under-expression of a target gene;
(b) subjecting said mammal to a pre-selected perturbance causing a desired physiologic stress in the mammal, and
(c) thereafter evaluating the responses of said genetically engineered mammal by determining the metabonomic profile of the mammal wherein said metabonomic profile is indicative of an adverse response to a drug effecting a target in conjunction with the pre-selected perturbance.

The invention also comprises a process for determining adverse responses to drugs against a target gene comprising:
a.) comparing the responses of at least two groups, each being of substantially identical non-human mammals, one of which is composed of genetically engineered mammals which exhibit either over-expression or under-expression of said target gene and the other of which exhibit substantially normal expression, that is, neither substantial over-expression nor under-expression, of said target gene;
b.) subjecting said mammals to substantively identical, pre-selected perturbances which cause a desired physiological stress in said mammals;
c.) determining the metabonomic profiles of said mammals and
d.) thereafter comparing said profiles to evaluate the adverse responses related to expression of the target gene and the preselected pertubances.

In addition to the foregoing, the invention includes as an additional aspect, all embodiments of the invention narrower in scope in any way than the variations specifically mentioned above.

### DETAILED DESCRIPTION OF THE INVENTION

The foregoing is provided to further facilitate understanding of the applicant's invention but is not intended to limit the scope of applicant's invention.

Definitions

The term “genetically engineered non-human mammal” (sometimes referred to below as an “engineered animal” for convenience sake) refers to all members of the class Mammalia except humans whose genome has been altered by human intervention so as to alter the expression level or pattern of a specific predetermined gene product. The genetically engineered non-human mammal utilized in this invention include, but are not limited to farm animals (pigs, goats, sheep, cows, horses, rabbits and the like), rodents (such as rats and mice), and domestic pets (for example, cats and dogs). Rodents are sometimes preferred because of their small size.

The term “genetically engineered non-human mammal” encompasses both knockout and transgenic animals which alter the level of expression of a particular gene product. Methods of genetic manipulation of mammals to alter gene expression are well known in the art. It also includes non-human mammals in which the temporal or spatial control of a specific predetermined gene product has been altered as described further below.

Nucleic molecules can be introduced into embryos by a variety of means to produce engineered animals. For instance, totipotent or pluripotent stem cells can be transformed by microinjection, calcium phosphate mediated precipitation, liposome fusion, retroviral infection or by other means. The transformed cells can then be introduced into embryos and incorporated therein to form engineered animals. In one method, developing embryos can be infected with retroviral vectors and engineered animals can be formed from the infected embryos. In another method, however, the DNA molecules of the invention are injected into embryos, preferably at the single-cell stage, which are allowed to develop into mature engineered animals. However, the invention is not limited to any of these methods but other methods of making engineered animals can be used as described, for example, in Transgenic Animal Generation and Use by L. M. Houdebine, Harwood Academic Press, 1997. Engineered animals also can be generated using methods of nuclear transfer or cloning using embryonic or adult cell lines as described for example in Campbell et al., Nature 380: 64-66 (1996) and Wilmut et al., Nature 385: 810-813 (1997). Further a technique utilizing cytoplasmic injection of DNA can be used as described in U.S. Pat. No. 5,523,222.

The term “determining the metabonomic profile” of an engineered animal refers to a procedure of determining the pattern of trace molecules in a biofluid obtained from the animal typically using high-resolution ¹H nuclear magnetic resonance (NMR) or mass spectroscopy, coupled with pattern recognition technology. The technique has been described by Robertson et. al. Biofluids upon which the technique is employed include but are not limited to urine, milk, plasma and serum We describe here a process in which engineered animals are exposed to a panel of perturbations and their response to the perturbations is quantitatively assessed by metabonomic profiling analysis of urine, serum, or plasma.

During target validation, an early stage of drug development, engineered animals (often rodents) are used to mimic the effects of drugs and evaluate drug safety.(Brayton, et. al.; Sundberg, et. al.; Ward, et. al.) For example, knockout mammals, i.e. mammals in which a specific gene is deleted, can be used to predict the effects of an inhibitory drug that prevents the function of a specific gene product (the target). That is, a knockout mammal in which the target gene of interest is deleted is a model to predict the effects, including adverse side effects, of an animal given a drug to inhibit the target molecule. Similarly, transgenic non-human mammals that overexpress a specific gene can be used to predict the effects of an agonist drug that causes increased function of a specific gene product. Additionally, engineered animals can be made that either under- or over-express the gene of interest only at certain times (temporal control) or in certain organs or tissues (spatial control) (Tymms, et al.). The advantage of using engineered animals for this purpose is that they can be examined and tested even before efficacious compounds or drugs have been synthesized.

Importantly, however, this conventional method of analyzing or “phenotyping” engineered animal is done in otherwise healthy, unstressed animals. Results from these tests may not predict safety in humans, whom often suffer from multiple diseases and a variety of stresses, nutritional problems, and adverse life style choices.

We propose two approaches to obtain better information about drug effects from engineered animals.
1. By perturbing or challenging the engineered animal with various agents or conditions instead of examining the non-human mammals only in an unperturbed state. For example, low doses of lipopolysaccharide (LPS), a component of the outer membrane of Gram-negative bacteria, simulate many of the effects of bacterial infection including fever and inflammation. Many of the physiologic responses to psychological stress can be evoked by animal behavior challenges, including exposure to flashing strobe lights, reversal of light:dark cycles by turning on the lights during the night and turning off the lights during the day, restraint for 20 minutes in a plastic tube, exposure to the odor of a cat or a rat, and foot shock for 10 seconds (Griebel, et. al.). Oxidative stress, a common effect of concurrent drug therapy, certain nutritional inadequacies, or illness, can be induced by feeding of buthionine sulfoximine, a compound that inhibits glutathione (Robertson, et. al.). Viral infections can be tested using experimental infection with a highly attenuated strain of influenza that is not pathogenic to humans and normally induces only minimal lesions in the mouse (Warren, et. al.).
2. Some alterations only have adverse effects in individuals with genetic predispositions to disease. For example, individuals vary in susceptibility to oxidative stress, epileptic seizures, and bacterial or viral disease. Effects of a drug on individuals with disease predisposition can be tested by breeding the engineered animals to strains with increased sensitivity to develop particular disorders. The progeny, then, will have both the original genetic modification (for example, deletion of the specific gene of interest) plus increased sensitivity to a particular disorder. Uncovering phenotypic abnormalities in these cross-bred non-human mammals may indicate potential adverse effects.

When expanding the battery of tests to be performed on engineered animals, it is highly desirable to accelerate the way the testing is evaluated. A rapid yet comprehensive way to evaluate responses is by metabonomics analysis. The advantage of metabonomics is that it simultaneously measures in a non-specific way all endogenous chemicals of a range of molecular sizes in biological fluids. Results are quantitative, can be compared among animals, and can be examined as a comprehensive pattern (by a process called “pattern recognition analysis”) rather than by individual chemical. Urine is collected by placing non-human mammals in metabolic cages, in which the urine is separated from feces and spilled food and diverted into a cooled collection vessel. Alternatively, serum or plasma could be used for metabonomics analyses. The body fluids are tested using ¹H-nuclear magnetic resonance (NMR) spectroscopy or mass spectroscopy. The spectra are analyzed by pattern recognition analysis and principal component analyses(Robertson, et. al.). An advantage of metabonomic analysis is that it is unbiased and broad. That is, it can measure changes in a wide variety of the body's endogenous chemicals in urine, serum, or plasma, even if those chemicals were not previously believed to be of interest.

It is expected that the metabonomic pattern of most strains of engineered animals will differ from wild-type (Gavaghan, et. al.). In the perturbation analysis, the difference between the metabonomic pattern after perturbation from the basal pattern will be assessed. Strains of engineered animals can be identified that have a metabonomic response to perturbation that is either (a) markedly increased or decreased from the metabonomic response of corresponding wild-type non-human mammals or (b) goes in a different direction from the metabonomic response of wild-type non-human mammals will be considered abnormal (see FIG. 1). The purpose of this screening test is to identify engineered animals with specific gene alterations that have abnormal responses to perturbations. This enables early prediction that drugs designed to inhibit or stimulate target proteins produced by a specific gene (i.e., the gene that is altered) may be unsafe in some humans under commonly encountered situations of physiologic perturbation. That is, the engineered animals will be used to model effects in individuals who are taking a drug at the same time that they are undergoing stress or perturbation.

Results of the screening test can then be followed by more detailed tests of particular analytes or mechanisms to better confirm or understand the results.

It will be clear that the invention may be practiced otherwise than as particularly described in the foregoing description and examples.

Numerous modifications and variations of the present invention are possible in light of the above teachings and, therefore, are within the scope of the invention.

The entire disclosure of all publications cited herein are hereby incorporated by reference.

1. A process for predicting adverse responses to drugs effecting a target by assessing the responses of animal models, comprising:
(a) providing a genetically engineered non-human mammal wherein said mammal exhibits either over-expression or under-expression of a target gene;
(b) subjecting said mammal to a pre-selected perturbance causing a desired physiologic stress in the mammal, and
(c) thereafter evaluating the responses of said genetically engineered mammal by determining the metabonomic profile of the mammal.

2. The method of claim 1 wherein the genetically engineered non-human mammal is a rodent.

3. The method of claim 1 wherein the evaluating is accomplished by comparing the metabonomic profile of the genetically engineered mammal with the metabonomic profile of a substantially identical non-engineered mammal which has been subjected to the same pre-selected perturbance.

4. The method of claim 1 wherein the metabonomic profile is determined using urine.

5. The method of claim 1 wherein the metabonomic profile is determined using serum.

6. The method of claim 1 wherein the metabonomic profile is determined using plasma.

7. The method of claim 1 wherein the metabonomic profile is determined using milk.

8. A method of claim 1 wherein the pre-selected perturbance is a low dose of lipopolysaccharide.

9. A method of claim 1 wherein the pre-selected perturbance is exposure to flashing strobe lights.

10. A method of claim 1 wherein the pre-selected perturbance is reversal of the light dark cycle.

11. A method of claim 1 wherein the pre-selected perturbance is restraint.

12. A method of claim 1 wherein the pre-selected perturbance is oxidative stress.

13. A method of claim 12 wherein the oxidative stress comprises feeding buthionine sulfoximine.

14. A method of claim 1 wherein the pre-selected perturbance is viral infection.

15. A method of claim 1 wherein the pre-selected perturbance is introduction of genetic material which results in increased sensitivity to develop particular disorders.
